SEQUENCE LISTING (4 sequences)

SEQ ID NO: 1 - DNA, 21 bp, synthetic construct
  source 1..21: mol_type = other DNA
gattactgctctggctcctag

SEQ ID NO: 2 - DNA, 21 bp, synthetic construct
  source 1..21: mol_type = other DNA
gactcatcgtactcctgcttg

SEQ ID NO: 3 - DNA, 20 bp, synthetic construct
  source 1..20: mol_type = other DNA
gacctggctcagcagtatgg

SEQ ID NO: 4 - DNA, 20 bp, synthetic construct
  source 1..20: mol_type = other DNA
agcttccctgccagctgtat

(19) United States
(12) Patent Application Publication
(10) Pub. No.: US 2026/0115266 A1
(43) Pub. Date: Apr. 30, 2026

(54) COMPOSITIONS COMPRISING PROTEIN DISULFIDE ISOMERASE AND METHODS OF PRODUCTION AND USE THEREOF

(71) Applicant: Board of Regents for the Oklahoma Agricultural and Mechanical Colleges, Stillwater, OK (US)

(72) Inventor: Véronique A. LACOMBE, Stillwater, OK (US)

(21) Appl. No.: 19/372,879

(22) Filed: Oct. 29, 2025

Related U.S. Application Data
(60) Provisional application No. 63/713,133, filed on Oct. 29, 2024.

(51) Int. Cl. A61K 38/52; A61K 9/00; A61K 38/17; A61K 38/28; A61P 3/10

(52) U.S. Cl. CPC A61K 38/52; A61K 9/0043; A61K 38/1709; A61K 38/28; A61P 3/10

(57) ABSTRACT

Compositions are disclosed that include at least one protein disulfide isomerase or a biologically active fragment or variant thereof. Methods of producing and using the compositions are also disclosed. In particular (but not by way of limitation), methods are provided for reducing blood glucose, treating hyperglycemia, treating or reducing the occurrence of type 1 diabetes, treating or reducing the occurrence of type 2 diabetes, and/or treating or reducing the occurrence of type 3 diabetes (i.e., Alzheimer's Disease) by administering the composition that includes the at least one protein disulfide isomerase. The protein disulfide isomerase may be administered alone or may be administered simultaneously or sequentially with one or more additional active agents.

### CROSS REFERENCE TO RELATED APPLICATIONS

This application claims the benefit under 35 USC § 119 (e) of U.S. Provisional Application No. 63/713,133, filed Oct. 29, 2024. The entire contents of the above-referenced patent application(s) are hereby expressly incorporated herein by reference.

### STATEMENT OF GOVERNMENT SUPPORT

This invention was made with government support under Grant No. K01RR023083-01 awarded by the National Institutes of Health. The government has certain rights in the invention.

### REFERENCE TO SEQUENCE LISTING SUBMITTED ELECTRONICALLY

The instant application contains, as a separate part of the present disclosure, a Sequence Listing which has been submitted via EFS-Web in computer readable form as an XML file. The Sequence Listing, created Oct. 27, 2025, is named “57910.262 Sequence Listing.xml” and is 4,643 bytes in size. The entire contents of the Sequence Listing are hereby incorporated herein by reference.

### BACKGROUND

Diabetes is a significant risk factor for developing cardiac dysfunction [1]. Sustained hyperglycemia during type 1 diabetes results from a lack of insulin production, which causes a failure of glucose uptake by insulin-sensitive tissues (striated muscle and adipose tissue). Translocation of glucose transporter 4 (GLUT4) from its intracellular vesicle to the cell surface is the rate-limiting step of insulin-mediated glucose uptake. Additionally, a novel class Ill glucose transporter, GLUT8, has recently been demonstrated to be an insulin-mediated GLUT isoform in the heart [2,3]. GLUT trafficking is regulated by other mechanisms, such as calcium and nitric oxide (NO), but these insulin-independent pathways are relatively under-investigated [4,5].

The sarco(endo)plasmic reticulum Ca²⁺ transport ATPase (SERCA) pump transports calcium ions from the cytoplasm into the sarcoplasmic reticulum allowing muscle relaxation and regulates intracellular Ca²⁺ homeostasis in animal and plant cells [6,7]. Cardiac-specific SERCA1a expression has been demonstrated to be partially protective against hyperglycemia in type 1 diabetic mice by increasing cardiac glucose uptake and GLUT trafficking in peripheral insulin-sensitive tissues (i.e. skeletal muscle and adipose) [5]. In order to determine how cardiac-specific SERCA overexpression modulates whole-body glucose homeostasis, quantitative mass spectrometry of transgenic heart was performed to identify potentially secreted proteins. Prolyl-4-hydroxylase subunit beta (P4hb) gene was up regulated by over 1800% in the TG ventricle and was identified as potential secreted proteins, based on SignalP [8]. The P4hb gene can be alternatively expressed; however, it is most likely being expressed as a protein disulfide isomerase (PDI). At the basic level, PDI does exactly what its name suggests by facilitating disulfide bond folding of newly synthesized proteins in the ER. However, PDI is a multifunctional protein that has been identified as part of the unfolded protein response (UPR) [9] and as a glucose response protein (GRP). The role of PDI in modulating whole-body glucose metabolism is unknown [10].

O-linked-N-acetylglucosaminylation (O-GlcNAc) is a post translational modification with a growing field of signals: regulating protein-protein interactions, altering protein structure or enzyme activity, changing protein subcellular localization, and modulating protein stability and degradation, such as apoptosis, the cell cycle, and stress responses, transcription, and epigenetics [11,12]. O-GlcNAc is almost exclusively found on nuclear and cytoplasmic proteins rather than membrane proteins and secretory proteins [11]. O-GlcNAcylation is dependent on the hexosamine pathway, which produces UDP-GlcNAc, which is O-linked to a protein by O-GlcNAc transferase, and removed by O-GlcNAcase. Over 5,000 proteins have been found to undergo this modification to date [13]. The hexosamine pathway is able to decrease insulin sensitivity, which is currently thought to occur for nutrient sensing. The molecular mechanism in vivo seems disputed; however, in vitro increased amounts of global O-GlcNAc dampens the amount of phosphorylation able to occur in the insulin signaling pathway (possibly through competitive inhibition), contributing to insulin resistance [14,15]. Hyperglycemia, hyperinsulinemia, and hyperlipidemia have all been shown to increase the amount of O-GlcNAcylation that occurs. Along with diabetes, dysregulation of O-GlcNAc has also been implicated in many pathologies, including Alzheimer's disease, cancer, and neurodegenerative disorders [16]. O-GlcNAcylation of the insulin signaling pathway specifically appears to be in order to inhibit glucose uptake in abundant amounts of UDP-GlcNAc [17,18]. Possible molecular links between hyperglycemia and arrhythmia have been identified as O-GlcNAcylation of CaMKII, causing dysfunctional calcium signaling [19].

Nitric oxide synthase (NOS) is the enzyme responsible for the production of nitric oxide (NO) in the body. Three isoforms of NOS-endothelial (eNOS), neuronal (nNOS), and inducible (iNOS)—are regulated via varying mechanisms, and all isoforms utilize L-arginine to produce NO and citrulline [20]. Ca²⁺ is a cofactor for both endothelial and inducible nitric oxide synthase (eNOS and iNOS). The NO produced by the various NOS isoforms have different reactivities, which alternatively modulate biological function [21]. Furthermore, these NO metabolites can modulate glucose metabolism [22]. For example, iNOS produces the greatest amount of peroxynitrite (ONOO⁻), which can impair glucose uptake [23].

Diabetes, defined by a sustained state of hyperglycemia, is an epidemic disease, affecting over 350 million people worldwide. In the United States alone, 9.3% of people suffer from this insidious disease. As many as 1 in 3 American adults are predicted to have diabetes by 2050, if current trends continue. Importantly, diabetes is a major and independent risk factor for cardiovascular diseases, including heart failure, heart attack, arrhythmia, and stroke. Therefore, there is a need in the art for new and improved treatments to help improve the conditions of millions of diabetic people worldwide.

### BRIEF DESCRIPTION OF THE DRAWINGS

FIG. 1. Transgenic hearts had increased glucose oxidation with rescued ATP production and cardiac efficiency. Mean±SE of cardiac work (A), glucose oxidation (B), palmitate oxidation (C), ATP production (D), cardiac efficiency (E), and oxygen consumption (F). *p<0.05, by One-Way ANOVA; #, p<0.05, by t-test vs the corresponding group without insulin, respectively. n=4-5, a, p<0.05, by t-test vs WTDX; b, p<0.05, by t-test vs the corresponding group without insulin. WTCT=wild-type control, TGCT=transgenic control, WTDx=wild-type diabetic, TGDx=transgenic diabetic.

FIG. 2. Transgenic mice had increased P4hb mRNA in the heart and greater protein in the serum. Mean±SE fold change of mRNA expression of P4hb is increased in transgenic mice (A), and increased P4hb protein in the serum (B) over their wild type counterparts. Statistical test: ΔΔCt method and two-tailed t-test. n=6-9/group, #p<0.05 vs WTC, *p<0.05 vs WTDx.

FIG. 3. Exogenous protein disulfide isomerase reduces blood glucose concentration in healthy and diabetic mice, without a change in insulin concentration. Mean±SE of blood glucose concentrations of healthy (A) and diabetic (B) mice vs exogenous PDI-treated counterparts; Insulin concentration of control vs type 1 diabetic (T1Dx) and T1Dx mice treated with PDI (C). n=5-6/group, #p<0.05 vs control, *p<0.05 vs diabetic.

FIG. 4. Exogenous protein disulfide isomerase rescues GLUT4 trafficking in type 1 diabetic ventricle and skeletal muscle. Top panel: representative Western blot of labeled/cell-surface GLUT4; bottom panel mean±SE of relative cell-surface GLUT4 expression of ventricle (A) and skeletal muscle (B). n=5-6/group, #p<0.05 vs control, *p<0.05 vs diabetic.

FIG. 5. O-GlcNAc levels in the ventricle were not significantly altered by T1Dx or the TG heart. Left panel: representative blot; right panel: mean±SE of relative total O-GlcNAc (A), GFAT1 (B), GFAT2 (C), OGT (D), and OGA (E) protein expression. GFAT=Glutamine fructose-6-phosphate amidotransferase, OGT=O-GlcNAc transferase, OGA=O-GlcNAc-ase. n=7-9/group, *p<0.05 vs diabetic.

FIG. 6. The hexosamine pathway was not different in skeletal muscle of TG or PDI-treated mice. Mean±SE of relative total O-GlcNAc (A), GFAT1 (B), GFAT2 (C), OGT (D), and OGA (E) protein expression. n=7-9/group, *p<0.05 vs. WTDx. GFAT=Glutamine fructose-6-phosphate amidotransferase, OGT=O-GlcNAc transferase, OGA=O-GlcNAc-ase.

FIG. 7. TG and PDI treatment rescued diabetes-induced reactive oxygen species (ROS) production in atrium. Mean±SE of ROS in the atrium. n=4-9/group, #p<0.05 vs. WTC, *p<0.05 vs. WTDx.

FIG. 8. Total ROS trending rescue in TG and PDI-treated ventricles. Mean±SE of ROS in the ventricle. n=4-9/group, #p<0.05 vs. WTC, *p<0.05 vs. WTDx.

FIG. 9. TG atrium had less nitric oxide and PDI-treatment inhibited iNOS. Mean±SE of total nitric oxide, nitric oxide from nNOS, iNOS, and eNOS origins, for TG vs WT (A) and diabetic vs PDI-treated (B) atrium. n=4-9/group, #p<0.05 vs. WTC, *p<0.05 vs. WTDx; nNOS=neural, iNOS=inducible, eNOS=endothelial, L-NNMA inhibits all Nitric Oxide Synthases.

FIG. 10. Total NO rescued in TG and PDI-treated ventricles. Mean±SE of NO in the ventricle. n=4-9/group, #p<0.05 vs. WTC, *p<0.05 vs. WTDx.

FIG. 11. PDI improves glucose transport in transgenic animals via a nitric oxide (NO)-dependent pathway.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another.

The terms “peptide” and “polypeptide” as used herein will be understood to refer to a polymer of amino acids. The polymer may include d-, l-, or artificial variants of amino acids. In addition, the terms “peptide” and “polypeptide” will be understood to include peptides, proteins, and glycoproteins.

The term “pharmaceutically acceptable” refers to compounds and compositions which are suitable for administration to humans and/or animals without undue adverse side effects, such as (but not limited to) toxicity, irritation, and/or allergic response, commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent to a subject/patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “pharmaceutically acceptable carrier or excipient” includes any carriers or excipients known in the art may be utilized in accordance with the present disclosure. For example (but not by way of limitation), a physiological compatible carrier (e.g., saline) that is compatible with maintaining the structure/activity of the active ingredient(s) when administered, and compatible with the desired mode of administration, may be utilized as the pharmaceutically acceptable carrier in accordance with the present disclosure. In addition, the active ingredient(s) may be mixed with excipients which are pharmaceutically acceptable and compatible with the active ingredient(s). Suitable excipients include, for example but not by way of limitation, water, saline, dextrose, glycerol, ethanol, and the like, or any combination thereof.

The term “effective amount” refers to an amount of a biologically active molecule or sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of infection by or growth of microbes and/or opportunistic infections. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the disease/infection in conjunction with the compositions of the present disclosure. This concurrent therapy can be sequential therapy, where the patient is treated first with one composition and then the other composition, or the two compositions are given simultaneously.

The terms “administration” and “administering,” as used herein, will be understood to include all routes of administration known in the art, including but not limited to, oral, topical, transdermal, parenteral, subcutaneous, intranasal, intratracheal, intrabronchial, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concepts, certain non-limiting embodiments of the present disclosure are related to compositions, systems, and kits that include protein disulfide isomerase (PDI), as well as methods of producing and using said compositions. The present disclosure is also related to compositions, systems, kits, and methods for reducing blood glucose in a patient, as well as methods of treating or reducing the occurrence of hyperglycemia or diabetes in a patient, utilizing the compositions comprising protein disulfide isomerase.

Certain non-limiting embodiments of the present disclosure are directed to a composition that comprises at least one protein disulfide isomerase or a biologically active fragment or variant thereof. Any protein disulfide isomerase or a biologically active fragment or variant thereof known in the art or otherwise contemplated herein may be utilized in accordance with the present disclosure. The term “protein disulfide isomerase” is used herein interchangeably with the terms “beta subunit of prolyl-4-hydroxylase” and “P4HB protein.”

The protein disulfide isomerase or a biologically active fragment or variant thereof may be produced by any methods known in the art or otherwise contemplated herein. For example (but not by way of limitation), the protein disulfide isomerase or a biologically active fragment or variant thereof may be isolated or purified from any mammalian tissue. Alternatively, in a particular (but non-limiting) embodiment, the protein disulfide isomerase or a biologically active fragment or variant thereof is recombinantly produced.

In certain particular (but non-limiting) embodiments, the composition may further include at least one additional active agent. Non-limiting examples of additional active agents that may be utilized in accordance with the present disclosure include Heat Shock Protein 90 (HSP90), insulin, and the like, as well as any combinations thereof.

Certain non-limiting embodiments of the present disclosure are also directed to pharmaceutical compositions for use in any of the methods disclosed or otherwise contemplated herein after, wherein any of the compositions described herein above is a pharmaceutical composition that comprises at least one protein disulfide isomerase. In certain particular (but non-limiting) embodiments, the pharmaceutical compositions further include at least one pharmaceutically acceptable carrier.

The phrase “pharmaceutically-acceptable carrier” as used herein means a pharmaceutically-acceptable material, composition, excipient, or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. The term “pharmaceutically-acceptable carrier” refers to any carrier, vehicle, excipient, and/or diluent known in the art or otherwise contemplated herein that may improve solubility, deliverability, dispersion, stability, and/or conformational integrity of the compositions disclosed herein. For example (but not by way of limitation), a physiological compatible carrier (e.g., saline) that is compatible with maintaining the structure/activity of the active ingredient(s) when administered, and compatible with the desired mode of administration, may be utilized as the pharmaceutically acceptable carrier in accordance with the present disclosure. In addition, the active ingredient(s) may be mixed with carriers/excipients which are pharmaceutically acceptable and compatible with the active ingredient(s). Suitable carriers/excipients include, for example but not by way of limitation, water, saline, phosphate-buffered saline (PBS), dextrose, glycerol, ethanol, and the like, or any combination thereof.

Any pharmaceutically-acceptable carriers known in the art or otherwise contemplated herein may be utilized in accordance with the present disclosure. Non-limiting examples include a pharmaceutically acceptable solvent, suspending agent, or vehicle that aid in delivery of the compositions of the present disclosure to the human or animal. The carrier may be liquid or solid and is selected with the planned manner of administration in mind. Examples of pharmaceutically acceptable carriers that may be utilized in accordance with the present disclosure include, but are not limited to, PEG, liposomes, ethanol, DMSO, aqueous buffers, oils, DPPC, lipids, other biologically-active molecules, vaccine-adjuvants, and combinations thereof. In addition, in certain particular (but non-limiting) examples, pharmaceutically-acceptable carriers can also contain a physiologically acceptable compound that acts to stabilize the compound and/or increase or decrease the absorption or clearance rates of the pharmaceutical compositions. Physiologically acceptable compounds can include (for example, but not by way of limitation) carbohydrates, such as glucose, sucrose, or dextrans; antioxidants, such as ascorbic acid or glutathione; chelating agents; low molecular weight proteins; detergents; liposomal carriers; and/or excipients or other stabilizers and/or buffers. Other physiologically acceptable compounds include (for example, but not by way of limitation) wetting agents, emulsifying agents, dispersing agents, and/or preservatives.

Certain non-limiting embodiments of the present disclosure are also directed to kits that comprise at least one of any of the compositions disclosed or otherwise contemplated herein (i.e., a composition comprising at least one protein disulfide isomerase). In addition, in certain particular (but non-limiting) embodiments, the kit comprises a first composition comprising at least one protein disulfide isomerase and a second composition comprising at least one additional active agent.

Non-limiting examples of additional active agents that may be utilized in accordance with the present disclosure include HSP90, insulin, and combinations thereof.

Certain non-limiting embodiments of the present disclosure are directed to methods that comprise administering one or more of any of the PDI-containing compositions disclosed or otherwise contemplated herein to a patient in need of treatment. The patient may be (for example, but not by way of limitation) a mammal, such as a human or a domestic animal.

The patient may have any condition for which treatment with one of the PDI-containing compositions of the present disclosure would be desired. Administration of the PDI-containing composition may have various results within the patient. For example (but not by way of limitation), the method may reduce blood glucose, improve glucose tolerance, and/or reduce insulin resistance in the patient or treat transient or sustained hyperglycemia in the patient. Alternatively (or in addition thereto), the method may be defined as a method of treating or reducing the occurrence of diabetes in a patient. For example, but not by way of limitation, the patient may have or be predisposed to have type 1 (insulin-deficient) diabetes, type 2 (insulin-resistant) diabetes, and/or type 3 diabetes. The term “type 3 diabetes” is used herein interchangeably with the term “Alzheimer's disease.”

In the methods of the present disclosure, the compositions may be administered in therapeutically effective amounts. An effective amount is a dosage of the composition sufficient to provide a therapeutically or medically desirable result or effect in the patient to which the composition is administered. The effective amount will vary with the particular condition being treated, the age and physical condition of the patient being treated, the severity of the condition, the duration of the treatment, the nature of the concurrent or combination therapy (if any), the specific route of administration, and like factors within the knowledge and expertise of the health practitioner. For example, in connection with methods directed towards treating patients having a condition characterized by elevated blood glucose or glucose intolerance, an effective amount would be an amount sufficient to mitigate, reduce, modulate, inhibit, or otherwise effectively treat said condition in the patient.

Generally, a therapeutically effective amount will vary with the patient's age, condition, and sex, as well as the nature and extent of the condition or disease in the patient, all of which can be determined by one of ordinary skill in the art. The dosage may be adjusted by the individual physician or veterinarian, particularly in the event of any complications. Non-limiting examples of therapeutically effective amounts that may be utilized in accordance with the present disclosure include about 0.01 μg/kg (weight of the at least one agent/body weight of patient), about 0.05 μg/kg, about 0.1 μg/kg, about 0.5 μg/kg, about 1 μg/kg, about 5 μg/kg, about 10 μg/kg, about 50 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 925 mg/kg, about 950 mg/kg, about 975 mg/kg, about 1000 mg/kg, about 1100 mg/kg, about 1200 mg/kg, about 1300 mg/kg, about 1400 mg/kg, about 1500 mg/kg, about 1600 mg/kg, about 1700 mg/kg, about 1800 mg/kg, about 1900 mg/kg, about 2000 mg/kg, and the like, as well as a range formed from two of any of the above values.

Non-limiting examples of ranges of therapeutically effective amounts that may be utilized in accordance with the present disclosure include a range from about 0.1 μg/kg to about 2000 mg/kg, or a range of from about 1 μg/kg to about 1000 mg/kg, or a range of from about 0.1 mg/kg to about 500 mg/kg, or a range of from about 0.5 mg/kg to about 100 mg/kg, or a range of from about 5 mg/kg to about 500 mg/kg, or a range of from about 100 mg/kg to about 500 mg/kg, or a range of from about 1 mg/kg to about 20 mg/kg, or a range of from about 5 mg/kg to about 50 mg/kg, or a range of from about 5 mg/kg to about 10 mg/kg, or a range of from about 0.01 mg/kg to about 5 mg/kg, or a range of from about 25 mg/kg to about 50 mg/kg, or a range of from about 0.1 mg/kg to about 1 mg/kg, or a range of from about 0.25 mg/kg to about 5 mg/kg, or a range of from about 10 mg/kg to about 200 mg/kg, or a range of from about 0.5 mg/kg to about 20 mg/kg, or a range of from about 10 mg/kg to about 60 mg/kg, and the like. The therapeutically effective amount may be delivered in one or more dose administrations. For example (but not by way of limitation, the therapeutically effective amount may be delivered in one or more dose administrations daily, for one or more days. If desired, the effective daily dose of the active compound may be administered as two, three, four, five, six, or more sub-doses, for example, administered separately at appropriate intervals throughout the day, optionally, in unit dosage forms. In some non-limiting embodiments, the agents/compositions are administered for more than 7 days, more than 10 days, more than 14 days, or more than 20 days. In still other non-limiting embodiments, the agent(s)/composition is administered over a period of weeks or months. In still other embodiments, the agent(s)/composition is delivered on alternate days. For example, the agent/composition may be delivered every two days, or every three days, or every four days, or every five days, or every six days, or every week, or every month, or similar.

Non-limiting examples of therapeutically effective daily amounts include amounts in a range from about 0.1 mg to about 1 g per daily total dose, or a range of from about 1 mg to about 500 mg per daily total dose, or a range of from about 5 mg to about 200 mg per daily total dose, or a range of from about 1 mg to about 100 mg per daily total dose, or a range of from about 5 mg to about 100 mg per daily total dose, or a range of from about 1 mg to about 75 mg per total daily dose, or a range of from about 5 mg to about 75 mg per daily total dose, or a range of from about 1 mg to about 60 mg per daily total dose, or a range of from about 5 mg to about 60 mg per daily total dose, or a range of from about 1 mg to about 50 mg per daily total dose, or a range of from about 5 mg to about 50 mg per daily total dose, or the like.

The compositions of the present disclosure may be administered alone or in combination with one or more additional therapies and may be administered by a variety of administration routes. The particular mode selected will depend, of course, upon the agent(s) selected, the condition being treated, the severity of the condition, whether the treatment is therapeutic or prophylactic, and the dosage required for efficacy. The methods of the present disclosure, generally speaking, may be practiced using any mode of administration that is medically acceptable, meaning any mode that produces effective levels of the active compounds without causing clinically unacceptable adverse effects. Non-limiting examples of administration routes that may be utilized in accordance with the present disclosure include oral, topical, transdermal, parenteral, subcutaneous, intranasal, intratracheal, intrabronchial, mucosal, intramuscular, intraperitoneal, intravitreal, and/or intravenous routes, and the like.

In certain particular (but non-limiting) embodiments, the probiotic compositions are formulated for oral, subcutaneous, and/or inhalant administration.

For example (but not by way of limitation), the composition can be administered to the pulmonary tract by any methods known in the art or otherwise contemplated herein. For example, but not by way of limitation, commercially available devices are known for many different methods and mechanisms of delivering various liquid or aerosolized pharmaceutical formulations to pulmonary tissue, including (but not limited to), intranasal instillation devices, intratracheal instillation devices, intratracheal injection devices, dry powder inhalers (DPIs), pressurized metered dose inhalers (pMDIs), nebulizers (such as, but not limited to, pneumatic (jet) nebulizers and electromechanical nebulizers), electrohydrodynamic aerosol devices, insufflators, respirators, and the like. These devices can include a single dose or multiple doses of the compositions of the present disclosure.

In addition, the formulations of the compositions of the present disclosure may include one or more additional components/elements that aid in the administration of the compositions, as based upon the delivery device. For example (but not by way of limitation), pMDIs, DPIs, and nebulizers typically employ one or more propellants to propel the liquid or cloud of dry powder formulation out of the device, to form an aerosol, and/or to atomize the liquid formulation. Any suitable propellants/pressurized gas supplies may be utilized. The propellant may take a variety of forms. For example, the propellant may be a compressed gas or a liquefied gas. Aerosol formulations for use in the subject method typically include (for example, but not by way of limitation) propellants, surfactants, and/or co-solvents. Suitable liquid compositions comprise the active ingredient in an aqueous, pharmaceutically acceptable inhalant solvent such as (but not limited to) isotonic saline or bacteriostatic water. Suitable liquid formulations for nasal sprays or nasal drops typically include (for example, but not by way of limitation) aqueous or oily solutions of the active ingredient.

When the compositions are formulated for being inhaled, the inhaled formulation may be designed for application to the upper (such as, but not limited to, the nasal cavity, pharynx, and larynx) and/or lower respiratory tract (such as, but not limited to, the trachea, bronchi, and lungs). Different devices and excipients can be used depending on whether the application is to the upper and/or lower respiratory tract and can be determined by those skilled in the art.

### EXAMPLES

Examples are provided hereinbelow. However, the present disclosure is to be understood to not be limited in its application to the specific experimentation, results, and laboratory procedures disclosed herein after. Rather, the Examples are simply provided as one of various embodiments and are meant to be exemplary, not exhaustive.

### Example 1

### Cardiac-Specific SERCA Overexpression Improves Cardiac and Systemic Glucose Metabolism During Diabetes

The heart is a very active metabolic organ which exerts endocrine effects by secreting cardiokines. However, its role in regulating whole-body metabolism has received scarce attention. It has previously been demonstrated that cardiac-specific overexpression of the sarcoplasmic reticulum calcium ATPase (SERCA) pump, which tightly regulates cytosolic calcium, modulates whole-body glucose homeostasis by enhancing glucose transport not only in the heart but also in insulin-sensitive tissue. Quantitative mass spectrometry of TG heart identified protein disulfide isomerase (PDI) as a cardiokine (p<0.0001). Therefore, this Example hypothesized that the cardiac SERCA pump could regulate glucose metabolism through PDI modulation.

To investigate how the cardiac SR Ca²⁺ ATPase (SERCA) pump and PDI modulate whole-body glucose metabolism, transgenic (TG) mice with cardiac-specific SERCA overexpression were used. Subgroups were made diabetic (Dx) by streptozotocin injections and compared to wild-type (WT) counterparts. Translocation of glucose transporters (GLUTs) was measured via a photolabeling biotinylation assay. Glucose and palmitate oxidation, cardiac work, and ATP production were quantified by retrograde perfusion in a working heart setting. Reactive oxygen species (ROS) and nitric oxide (NO) were measured using electron paramagnetic resonance.

Blood glucose was lower in transgenic diabetic (TG Dx) mice versus wild type diabetic (WT Dx) mice (p<0.05). Cardiac efficiency, glucose oxidation, and cardiac GLUT trafficking were decreased in WT Dx mice, which was restored in TG Dx mice (p<0.05). Although total O-GlcNAcylation levels (measured by Western blotting) were unchanged, glucosamine-fructose-6-phosphate aminotransferase, O-GlcNAc transferase, and O-GlcNAcase were upregulated in TG mice hearts. Protein disulfide isomerase (PDI) was upregulated in the serum of TG mice versus WT mice. Intranasal inoculation with exogenous PDI reduced blood glucose in wild type (WT) healthy mice and partially rescued hyperglycemia in WT diabetic mice compared to untreated counterparts (P=0.009 and P=0.044, respectively), without any change in insulin concentration. Furthermore, PDI treatment increased cell-surface GLUT4 translocation in both the heart and skeletal muscle of treated vs. untreated WT Dx mice (p<0.05). Both cardiac-specific SERCA overexpression and PDI treatment rescued Dx-induced ROS and total NO production. PDI treatment significantly inhibited NO production from the pro-inflammatory inducible nitric oxide synthase (iNOS). Taken together, these results indicate that the cardiac SERCA pump could regulate glucose metabolism through PDI and iNOS modulation and therefore provide a novel therapeutic target for diabetes.

### INTRODUCTION

The mechanism by which a cardiac-specific overexpression of SERCA1a could induce a recovery in whole-body glucose metabolism was unknown. This Example provides evidence that cardiac SERCA overexpression activated P4hb gene transcription and secretion; as such, increased PDI circulation is then able to disrupt iNOS activity leading to a recovery in glucose metabolism.

### Methods

Animal Model. A transgenic (TG) mouse model on an FVB/N background expressing the SERCA1a (skeletal isoform) in the heart, as previously described [5], in addition to the cardiac SERCA2a isoform, was utilized. Since the SERCA1a pump can substitute to the cardiac isoform, cardiac-specific SERCA1a expression resulted in increased total SERCA protein expression by ˜50% in the TG heart [5]. Tail tips were collected from each mouse upon weaning, which were used for genotyping via PCR reactions. Eight- to twelve-week old mice (male and female combined) were randomly selected to receive streptozotocin injection of 65-95 mg/kg every 48 hours in order to induce a type 1 diabetic condition. To monitor blood glucose levels weekly, mice were fasted for 4 to 6 hours, and venous blood was drawn from the facial vein. Blood was tested using a handheld glucometer (Bayer Contour, Tarrytown, NY). Upon the 8th week after the final STZ injection, tissue samples were collected from fasted mice for either proteomic analysis or biotinylation photolabeled analysis. 30 μg of exogenous PDI was dissolved in 20 μL of PBS and inoculated intranasally to lightly anesthetized mice. All procedures were approved by the Oklahoma State University Institutional Animal Care and Use Committee.

Isolated Working Heart. To evaluate the changes in heart function of TG vs WT mouse hearts, heart function was characterized ex vivo. After an intraperitoneal injection of pentobarbital, hearts were excised and perfused using a modified Krebs-Henseleit with added glucose (5 mM) and palmitate (0.5 mM) bound to BSA (3%) [24]. Cardiac temperature was maintained at 37° C., and data were obtained and analysed using LabChart 7Pro software (ADInstruments, Bella Vista, Australia). The hearts were perfused in the Langendorff mode [25] where metabolic substrate was added, and flow through metabolites were measured, as previously described [26].

Quantitative Real-time Polymerase Chain Reaction Analyses (qRT-PCR). Whole heart samples (50-80 mg) were pulverized using Trizol reagent (Invitrogen, CA, USA) according to the manufacturer's instructions and RNA quantified via absorbance (A260) using Gen5 software with Biotek synergy HT hardware on a take3 plate (BioTek, VT, USA). Purity was assessed via the A260/A280 ratio. DNAse-treated RNA (Ambion AM1906M) was then converted to complementary DNA using the High-Capacity cDNA Reverse Transcription Kit and random primers (Applied Biosystems, CA, USA) according to manufacturer's recommendations and stored at −80° C. until analysis. The qRT-PCR assays for relative quantification of β-actin, vinculin, talin-1, cadherin-13, heat shock protein 90, fibrinogen beta, and alpha-2-macroglobulin were performed using SYBR Green Real Time PCR Master Mix containing AmpliTaq Gold DNA Polymerase, to minimize nonspecific product formation, and deoxyribose nucleotide triphosphates with deoxyribose uridine triphosphate, to reduce carryover contamination (Applied Biosystems). Primer sequences were selected using the National Center for Biotechnology Information Basic Local Alignment Search Tool (NCBI BLAST) and custom synthesized by Invitrogen (GenBank accession numbers: β-actin: NM_007393.5; Prolyl-4-hydroxylase beta subunit (P4Hb): NM_011032.3. Primer Sequences: β-actin: forward-GAT TAC TGC TCT GGC TCC TAG (SEQ ID NO:1), reverse-GAC TCA TCG TAC TCC TGC TTG (SEQ ID NO:2), P4Hb: forward-GAC CTG GCT CAG CAG TAT GG (SEQ ID NO:3), reverse-AGC TTC CCT GCC AGC TGT AT (SEQ ID NO:4)). Each PCR reaction (20 μL) contained 2× reaction buffer (SYBR Green I dye, Amplitaq Gold DNA Polymerase, deoxyribose nucleotide triphosphates with deoxyribose uridine triphosphate, passive reference, and optimized buffer components), forward and reverse primers (0.5 mM), 0.5 μg of complementary DNA, and DNase-RNase-free water. Primer concentrations and PCR conditions were determined during initial optimization runs. Samples were run in duplicate in a 96-well MicroAmp optical plate (Invitrogen). qRT-PCR was performed in an ABI 7500 Fast instrument (Applied Biosystems) with the following cycling conditions: 10 minutes at 95° C., followed by 40 cycles at 95° C. for 15 seconds, and 60° C. for 1 minute. No-template, negative controls were included for each gene. A melting curve was generated to ensure product purity and the absence of primer dimers. Primers were further confirmed via amplification. The messenger RNA (mRNA) expression of target genes was normalized to β-actin, and relative gene expression was quantified using the ΔΔCT method [27].

ELISA. Serum was collected from mice during sacrifice. Whole blood was collected and allowed to coagulate for 30 mins RT. Samples were centrifuged at 2,000 g for 10 mins at 4° C. Supernatant was collected and frozen until analysis by ELISA. Mouse P4hb and insulin ELISA's were obtained (MBS933098 MyBioSource, Ca, USA; and ab277390 ABCAM, Cambridge, UK) and performed as directed.

Photolabeling Biotinylation Assay. Following a 1-hour Langendorff perfusion, atria and ventricles were photolabeled with the cell surface impermeant biotinylated bis-glucose photolabeling reagent (bio-LC-ATB-BGPA, 300 μM, Toronto Research Chemicals, ON, Canada), of which the hexose group interacts specifically with the extracellular binding site of GLUTs, as previously by our group [28]. Similarly, skeletal muscle and adipose tissue were photolabeled following mincing and a 1-hour incubation in Krebs-Henseleit buffer. After infusion of the photolabeled reagent through the aorta (in the case of the atria and ventricles) or into the buffer, also at a concentration of 300 μM (in the case of the skeletal muscle and adipose tissue), the photolabeled reagent was cross-linked to cell-surface GLUTs using a Rayonet photochemical reactor (340 nm, Southern New England UV), as previously described [2,3].

From these, membrane enriched protein extracts were obtained by homogenizing tissue samples in a buffer containing 210 mM sucrose, 40 mM NaCl, 2 mM EDTA, 30 mM HEPES, and 2% protease inhibitor cocktail (Sigma, St. Louis, MO). The homogenate was subsequently incubated in a buffer containing 58 mM sodium pyrophosphate and 1.17 mM KCl. The membrane fraction was recovered via homogenization and ultracentrifugation at 40,700 g for 90 minutes at 4° C., at which point the collected pellet was re-suspended in a buffer containing RIPA and 0.2% protease inhibitor cocktail, and rotated for 1 hour at 4° C. Following this, the samples were centrifuged at 3,000 g for 30 minutes at 4° C. The supernatant was stored at −80° C. for further analysis.

Equal amounts of protein (5-20 μg) were resolved in a 12% SDS-polyacrylamide gel and electrophoretically transferred to a polyvinyl-idine fluoride membrane (BioRad), as previously described [2,3]. After incubating in blocking buffer (5% non-fat dry milk) for one hour, membranes were incubated with primary antibodies overnight (anti-human GLUT4, 1:750, AbD Serotec; anti-human GLUT8, 1:500, Bioss) followed by a 1-hour incubation of appropriate secondary antibody conjugated to horseradish peroxidase (polyclonal donkey anti-rabbit, 1:2500, GE Healthcare). Primary antibodies were chosen based on their 100% sequence homology with the protein of interest in rodents, and validated against a positive control (i.e., tissue, peptide). Antibody-bound transporter proteins were quantified by enhanced chemiluminescence reaction (KPL). Band density was quantified using GelPro Analyzer (Media Cybernetics). The data was expressed relative to appropriate controls.

Western Blotting. LV samples were homogenized in 3 mL of Ripa buffer for 1 g of tissue plus 1×PMSF (Interchim, Montluçon, France) and 2× protease inhibitor cocktail (Roche, Mannheim, Germany). Protein samples (25 μg) were submitted to electrophoresis on a 10% polyacrylamide/sodium dodecyl sulfate gel and then were run 150 min at 20 mA per membrane in TG-SDS buffer (Interchim). Gels and nitrocellulose membranes (Hybond C super membrane, Amersham, Saclay, France) were equilibrated in TG-SDS buffer with 20% ethanol, and protein fractions were transferred using an electroblotting apparatus (Bio-Rad, Marnes La Coquette, France). Nonspecific binding was blocked by incubating membranes in 5% non-fat dry milk in Tris-buffered saline (TBS) (200 mM Trizma base, 1.4 M NaCl, pH=7.5) with 0.1% Tween 20 added (TBS-T), and then membranes were incubated with the primary antibody: O-GlcNAc, GFAT1, GFAT2, OGT, and OGA (#9875, #5322, #6917, #24083, #60406, Cell Signaling, Ma, USA). Membranes were washed in 5% non-fat dry milk in TBS-T and hybridized with the secondary antibody in 1% non-fat dry milk in TBS-T. Finally, membranes were washed with 5% non-fat dry milk in TBS-T then TBS, and antibody complexes were revealed by the enhanced chemiluminescence detection process (Bio-Rad). Chemiluminescence was visualized using an Amersham ImageQuant RT-ECL camera (GE Healthcare), and band signals were assessed by densitometry with ImageQuant TL software (GE Healthcare). For each lane, a ratio to the corresponding stain free intensity was calculated, as previously described [29].

Measurement of NO and O₂. Atria and ventricle samples were collected from mice and immediately placed into 37° C. Krebs buffer with respective NOS inhibitors. After 30 mins, Krebs buffer was aspirated, and spin trap buffer was added. Samples were incubated at 37° C. for 45 minutes. Atrium and ventricle samples were placed in syringes with ˜100 μL of spin trap buffer and flash frozen for measurement of NO and O₂ by electron paramagnetic resonance (EPR), as previously described [30].

Statistical Analysis. Shapiro-Wilk and Levene's test were used to assess the normality and homogeneity of Western blot data, respectively. Differences between means were assessed using 1-way ANOVA with Student Newman Keuls post-hoc text and Student's t test, as appropriate. Statistical significance was defined as p<0.05. Data are reported as mean±standard error.

### Results

### Transgenic Diabetic Hearts Exhibited Increased Glucose Oxidation, ATP Production, and Cardiac Efficiency

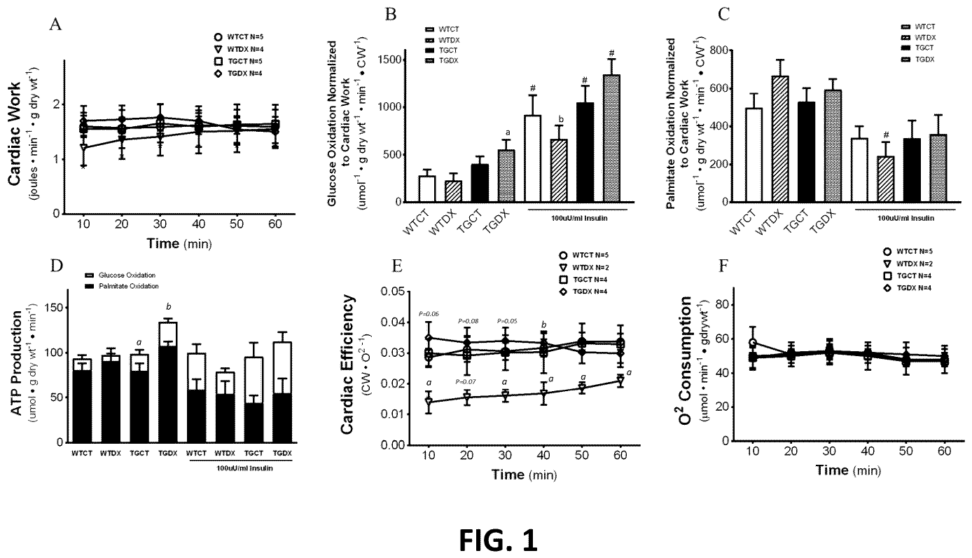

To further characterize the metabolic profile of TG hearts compared to the WT counterparts, an isolated working heart setting was utilized. While calcium handling was more efficient due to the overexpression of SERCA in the TG hearts, total cardiac work remained unchanged (FIG. 1, Panel A). Glucose oxidation was impaired in the wild-type diabetic (WTDx) hearts, while transgenic diabetic (TGDx) hearts had greater glucose oxidation with and without insulin (FIG. 1, Panel B). Similar findings were observed for palmitate oxidation with the WTDx being more reliant on fatty acid to maintain energy demands whereas TGDx hearts had restored glucose oxidation and surprisingly produced more ATP (FIG. 1, Panels C and D). Cardiac efficiency was reduced in the WTDx hearts, which was restored in the TGDx hearts without any difference in oxygen consumption between groups (FIG. 1, Panels E and F).

Transgenic Mice had Increased P4hb mRNA in the Heart and Greater Protein in the Serum

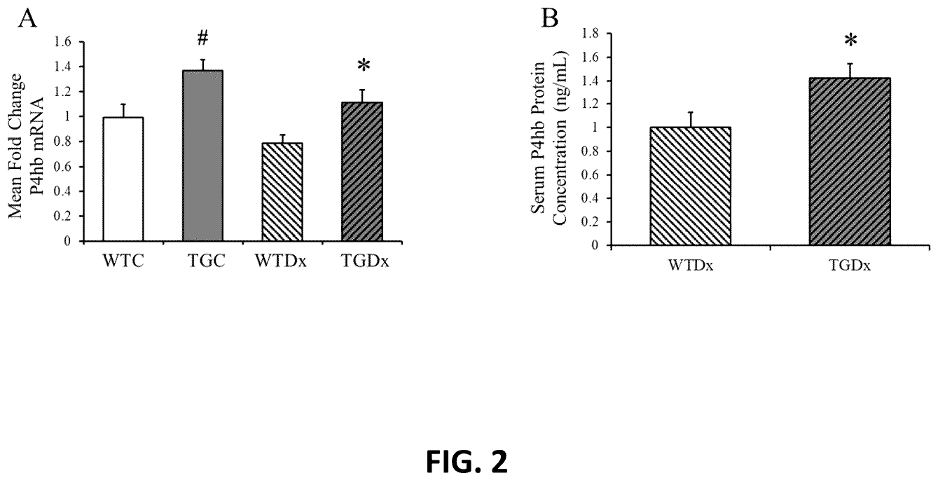

To confirm upregulation of P4hb RNA, which codes for prolyl-4-hydroxylase beta subunit, P4hb RNA levels of WT vs TG hearts were measured using qrtPCR. P4hb was upregulated in hearts of TGC and TGDx mice compared to their WT counterparts (FIG. 2, Panel A). To confirm that P4hb is a secreted cardiokine, an ELISA was performed on serum of these mice, P4hb was found to be significantly increased in serum of TGDx mice (FIG. 2, Panel B).

Exogenous Protein Disulfide Isomerase Reduces Blood Glucose Concentration in Healthy and Diabetic Mice Independent from Insulin Concentration

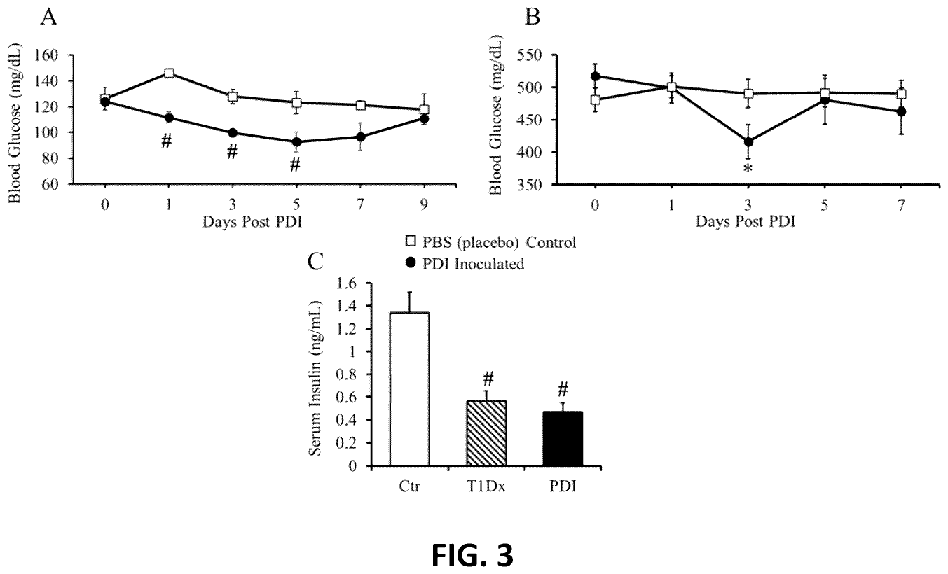

Since protein disulfide isomerase (PDI) is the most likely form of P4hb to be upregulated in serum, this Example determined if greater circulating PDI could modulate whole-body glucose metabolism. To this end, healthy and T1Dx mice were inoculated with exogenous recombinant PDI. Exogenous PDI decreased the blood glucose concentration of healthy mice over the course of 5 days (FIG. 3, Panel A). In addition, blood glucose of T1Dx mice was reduced at day 3 post inoculation (FIG. 3, Panel B). Since PDI aids in folding and secretion of insulin [31], it was tested whether reductions in blood glucose were due to increased serum insulin. Serum insulin concentration was similar in T1Dx mice before and after PDI treatment, indicating that PDI acts through an insulin-independent mechanism (FIG. 3, Panel C).

### Exogenous Protein Disulfide Isomerase Rescues GLUT4 Trafficking in Type 1 Diabetic Ventricle and Skeletal Muscle

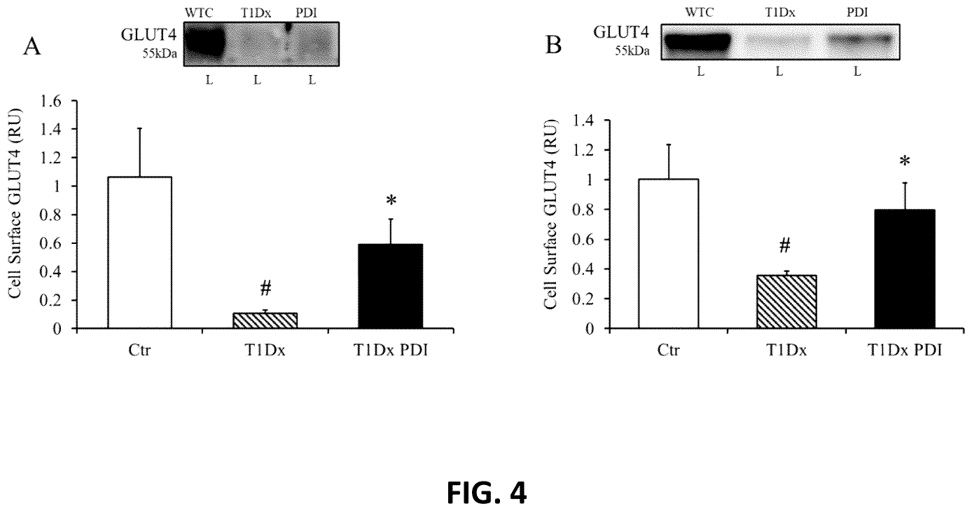

Next, this Example determined if exogenous PDI was able to recover glucose transport in striated muscle of T1Dx mice through an insulin-independent mechanism. Cell-surface GLUT4 was measured in the heart and skeletal muscle using a photolabeling biotinylation assay. T1Dx elicited a loss of cell-surface GLUT4 content in heart and skeletal muscle, while exogenous PDI treatment enhanced cell-surface GLUT4 trafficking of T1Dx hearts and skeletal muscle (FIG. 4, Panels A and B).

O-GlcNAc Levels in the Ventricle were not Significantly Altered by T1Dx or the TG Heart

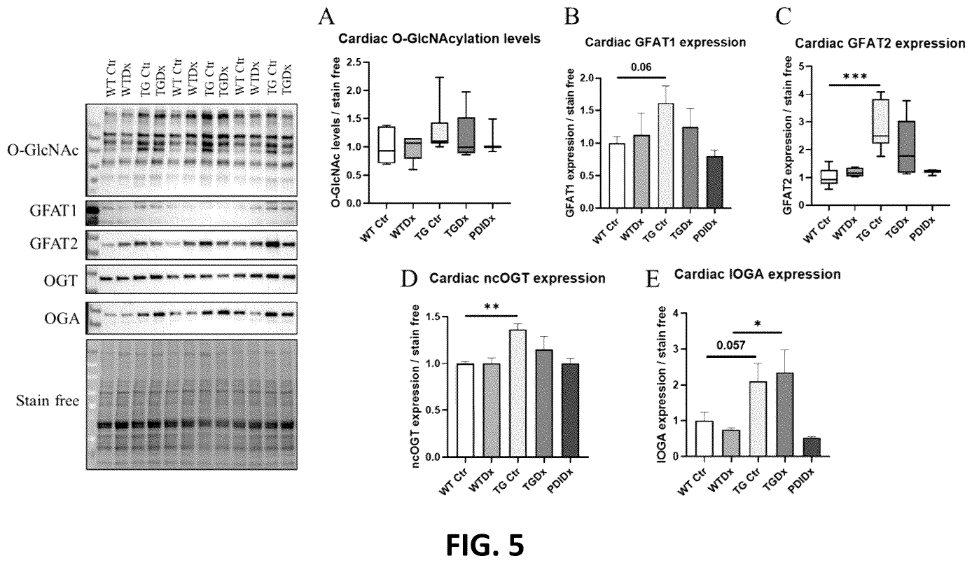

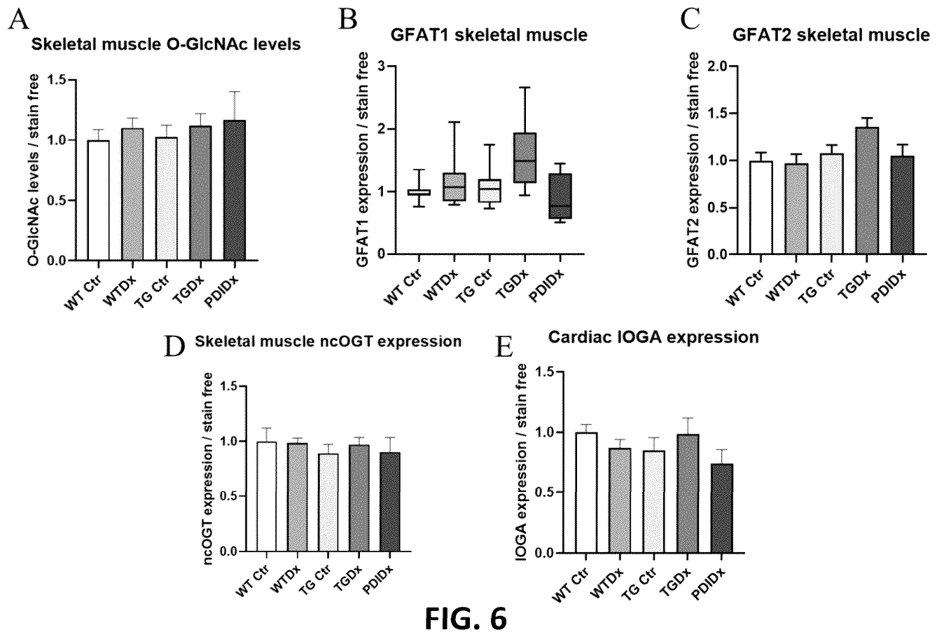

Next, the hexosamine pathway was analyzed, since it is linked to various metabolic diseases and modulates protein function by adding or removing the post-translational modification O-GlcNAc [32,33]. While total O-GlcNAc remained unchanged, GFAT1, GFAT2, OGT, and OGA protein expression were all increased in TGC hearts (FIG. 5, Panels A-E). OGA was the only protein upregulated in the TGDx compared to WTDx hearts (FIG. 5, Panel E). The hexosamine pathway was unaffected by PDI treatment (FIG. 5, Panels A-E). The same parameters were measured in the skeletal muscle; however, no significant changes were identified in the respective groups.

### TG and PDI Treatment Rescued Diabetes-Induced Reactive Oxygen Species (ROS) Production in the Atrium

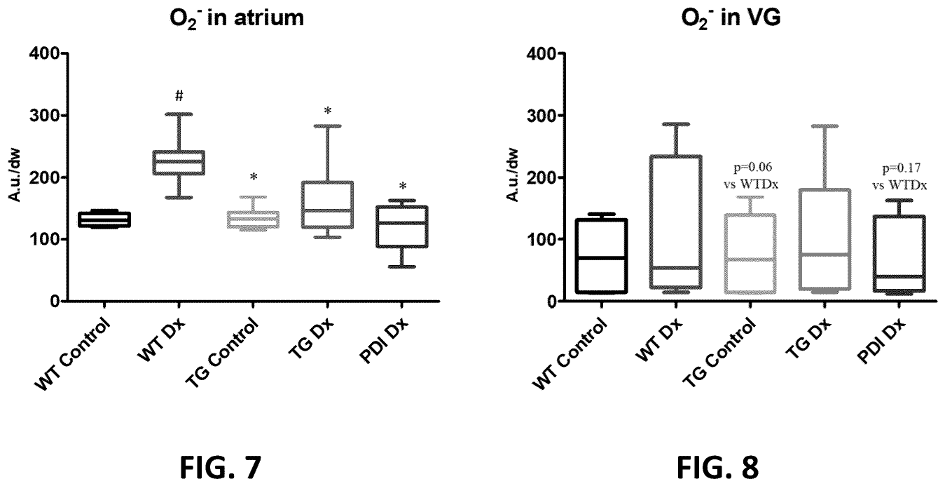

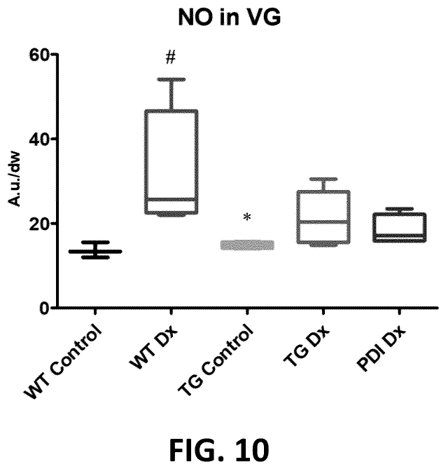

A detrimental side effect of diabetes is production of reactive oxygen species (ROS)³⁴. ROS was confirmed to be elevated in this WTDx model. Furthermore, ROS was found to be rescued in the atrium of both TGDx and PDI treated WTDx mice (FIGS. 7-8). In the ventricle, this pattern was significantly reduced (FIG. 10).

TG Atrium had Less Total Nitric Oxide and PDI Treatment Inhibited iNOS

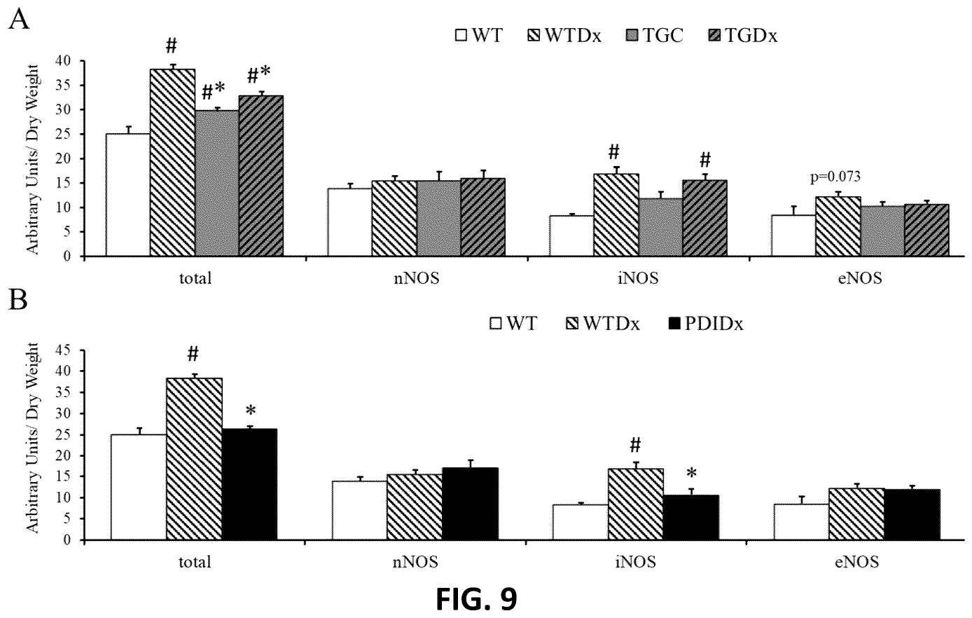

Another insulin-independent pathway that could increase glucose uptake is nitric oxide (NO) signaling [35]. Using electron paramagnetic resonance (EPR), it was determined that WTDx mice had greater total NO in the atrium, which was recovered in TGDx mice (FIG. 9, Panel A). Similarly, PDI treatment reduced NO levels back to control levels by inhibiting the inducible nitric oxide synthase (iNOS) (FIG. 9, Panel B). This rescue of total NO in TGDx and PDI treated WTDx remained consistent in the ventricle.

### DISCUSSION

While the incidence of diabetes and diabetic cardiomyopathy continue to rise, the relationship between calcium signaling and glucose uptake remains elusive. Previous investigations suggested mice with a cardiac-specific upregulation of SERCA in the heart are partially protected against hyperglycemia during type 1 diabetes [5]. It was also determined that SERCA1a overexpression increased GLUT4 trafficking in diabetic hearts and peripheral insulin-sensitive tissues, suggesting SERCA pump activation and/or enhanced intracellular calcium flux is a major regulator of glucose transport [5]. In this Example, it was confirmed that calcium flux rescued glucose oxidation and restored cardiac efficiency. Thus, this data indicated SERCA pump activation and/or enhanced intracellular calcium flux is a major regulator of glucose transport [5]. Furthermore, the results presented herein indicated the heart secretes proteins that confer whole-body protective effects in restoring glucose homeostasis in the face of type 1 diabetes.

While PDI largely serves as an ER chaperone for formation of disulfide bonds, it is a multifunctional protein [36] that plays a synergistic role with certain proteins upregulated in hearts of TG mice. PDI is also classified as a thyroid hormone binding protein [37] and modulator of thyroid hormone receptors [38]. As PDI, calnexin, and calreticulin are all ER chaperones, an underlying mechanism promoting secretion of unknown factors that rescued hyperglycemia in the TG mice. However, following PDI inoculation and subsequent reduction of blood glucose in healthy and T1Dx mice, the cardiokine responsible for the TG rescue in blood glucose concentrations was identified herein. This reduction in blood glucose was achieved due to the recovery of GLUT4 translocation in both the heart and skeletal muscle of PDI-treated mice.

The next aim of this Example was to identify a potential mechanism by which PDI reduced blood glucose concentrations. PDI acts directly on insulin; however, PDI can exhibit pro- or anti-insulin action [31,39]. Following measurement of serum insulin, a rescue of serum insulin levels was not identified in TG or PDI-treated mice. Therefore, reduction of blood glucose and recovery in cell-surface GLUT4 following PDI treatment apparently is insulin-independent.

Exploring alternative pathways, the hexosamine biosynthase pathway gained our interest, as it was identified as a post-translational modification with a wide array of applications [32,33,40], including glucose metabolism [41]. Prior to measurement of O-GlcNAc in samples, an up or down regulation of total O-GlcNAc that may regulate glucose metabolism was anticipated. Surprisingly, differences in total O-GlcNAc levels were not observed. However, increased expression of the regulatory proteins within the hexosamine pathway (i.e., GFAT, OGT, and OGA) in TG mice was detected. This indicates greater efficiency and turnover of O-GlcNAc residues. These alterations were only identified in the heart; the skeletal muscle did not exhibit significant differences in the hexosamine pathway, but a recovery in GLUT trafficking in TG and PDI-treated mice. Therefore, the rescue of whole-body glucose metabolism does not appear to be dependent on hexosamine pathway activation. Whether or not activation of this pathway in the heart aided with PDI expression or other cardiokines that induced whole-body effects is not known. Finally, activation of the hexosamine pathway was not observed in PDI-treated mice. Therefore, activation of this pathway was not simply a response to increased glucose metabolism. Rather, the hexosamine pathway was influenced by increased calcium flux of the TG mice. In summary, increased calcium recycling increased O-GlcNAc recycling.

PDI and HSP90 play key roles in a potential novel pathway involving nitric oxide, which increases GLUT trafficking in insulin-sensitive tissues [31]. Nitric oxide (NO) must be produced by nitric oxide synthase (NOS), and NOS activity is increased by HSP90 [42]. PDI is able to form s-nitrothiols to deliver NO throughout the blood stream [43]. Hence, the combined upregulation of these two proteins is predicted to increase cellular glucose uptake and reduce hyperglycemia during type 1 diabetes. Therefore, an increase of NO from endothelial NOS (eNOS) was anticipated. However, total NO levels were reduced due to inhibition of inducible NOS (iNOS). While this did not follow the previous hypothesis, iNOS is a pro-inflammatory NOS due to the largest production of peroxynitrites, which impairs physiological NO signaling and inhibits glucose uptake [23]. Furthermore, PDI modulates NOS function [44], and it seems PDI restored physiological NO signaling via the inhibition of iNOS. However, hyperglycemia is able to activate iNOS [45, 46]. Therefore, PDI could potentially increase glucose uptake via a different mechanism, and the reduced iNOS could be a result of the decreased blood glucose. Other than the possibility of direct inhibition of iNOS, exogenous PDI could potentially inhibit iNOS function through restoring the redox state of these mice [43].

Thus, this Example has determined that mice with a cardiac-specific overexpression of SERCA not only possess increased GLUT trafficking in the atria and ventricle, but also in peripheral insulin-sensitive tissues: for example, skeletal muscle or white and brown adipose tissues. It was determined that the TG heart was secreting novel proteins during diabetes, which facilitates or assists in return to normal whole-body glucose homeostasis in the face of type 1 diabetes. PDI was tested as one of these potential cardiokines, which increases glucose uptake by rescuing GLUT4 trafficking in striated muscle by an insulin-independent mechanism. PDI is then able to reduce ROS by inhibiting iNOS, thereby restoring glucose uptake.

### Example 2

Diabetes is an epidemic disease, affecting over 350 million people worldwide. It is defined by a sustained state of hyperglycemia, resulting from decreased glucose uptake into insulin-sensitive tissues (i.e., striated muscle and adipose tissue) due to a lack of insulin production (type 1) or action (type 2). As described in Example 1, a unique line of genetically altered (transgenic) mouse model with an upregulation of a calcium transporter protein in the heart (e.g., sarcoplasmic reticulum calcium ATPase pump) was utilized. Calcium is required for normal heart contraction and rhythm. Example 1 demonstrated that this transgenic mouse model is partially protected against diabetes. Proteomics analysis of the cardiac secretome of this transgenic mouse model revealed protein disulfide isomerase (PDI) as a potentially secreted cardiokine responsible for these metabolic effects. The PDI protein was further confirmed to be significantly up regulated in the serum of transgenic animals compared to wild type counterparts. Wild-type type 1 and type 2 diabetic mice treated with PDI exhibited a reduction in blood glucose and improved glucose tolerance, respectively, compared to their placebo-treated counterparts. Taken together, these results indicate that PDI provides a novel therapeutic approach for diabetic patients.

Example 1 demonstrated that cardiac-specific upregulation of the sarcoplasmic reticulum calcium ATPase (SERCA) pump, a ubiquitous protein that tightly regulates cytosolic calcium (Ca²⁺), rescues diabetes-induced alterations in cardiac glucose transport and improves whole-body glucose homeostasis in transgenic diabetic mice. Example 1 further demonstrated a substantial increase in the expression of protein disulfide isomerase (PDI) in the ventricle and serum of healthy and diabetic transgenic mice with cardiac-specific SERCA overexpression. PDI is a molecular chaperone activated by ER stress. Due to the substantial rescue in trafficking of glucose transporter (GLUT) 4 in cardiac, skeletal, and adipose tissues of transgenic diabetic mice compared to their wild type diabetic counterparts, PDI is also indicated to be a cardiokine candidate with endocrine effects on whole-body glucose metabolism. Following intranasal PDI administration, a significant reduction of blood glucose was observed in healthy and diabetic WT mice, as well as an improved glucose tolerance in type 2 diabetic mice.

The data presented herein has shown that intranasal administration of PDI significantly reduced blood glucose up to five days in wild type (WT) healthy and diabetic mice with insulin deficiency. It has further been demonstrated that PDI improved glucose tolerance in type 2 diabetic mice fed a high fat diet as well as in diabetic mice with partial deficiency of the insulin receptor substrate. While the scope of the present disclosure encompasses the administration of PDI by any methods disclosed or otherwise contemplated herein, the administration of PDI by less invasive methods than injection (such as, but not limited to, the innovative intranasal delivery approach described herein) reduces the burden of daily insulin injections for diabetic patients and thereby should increase treatment compliance.

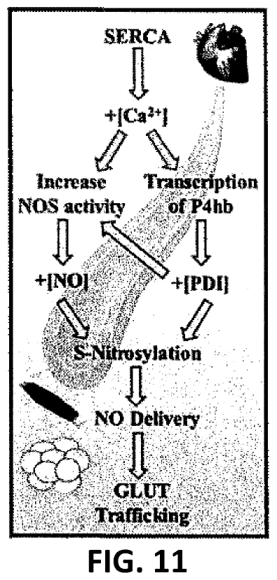

This Example indicates that PDI improves glucose transport in transgenic animals via a nitric oxide (NO)-dependent pathway (FIG. 11). PDI has not been described as a potential regulator of glucose metabolism in either healthy or diabetic subjects, and thus leads to a novel therapeutic strategy for diabetic patients.

Protein Disulfide Isomerase (PDI) treatment has been shown in Example 1 to not only reduce blood glucose concentration but to also improve glucose tolerance in type 1 and type 2 diabetic subjects. This provides a distinct advantage over currently-used insulin therapy. Additionally, this treatment likely does not act through insulin receptors. As type 2 diabetic patients suffer from chronically over-stimulated insulin receptors, this treatment that works through an insulin-independent pathway to reduce blood glucose thereby provides a particularly effective treatment for type 2 diabetes.

In addition, it has been further demonstrated that PDI reduces hyperglycemia in diabetic mice with partial deficiency of the insulin receptor substrate (IRS2 KO^{+/-}), indicating that PDI works independently of the insulin signaling pathway. In addition, PDI significantly increased glucose uptake in MDCK cells, which are considered insulin-independent cells. These data indicate that PDI treatment provides a novel therapeutic strategy for all diabetic patients that can be administered alone or in combination with insulin.

### NON-LIMITING ILLUSTRATIVE EMBODIMENTS

Illustrative embodiment 1. A pharmaceutical composition, comprising at least one protein disulfide isomerase or a biologically active fragment or variant thereof; and at least one pharmaceutically acceptable carrier.

Illustrative embodiment 2. The pharmaceutical composition of Illustrative embodiment 1, wherein the at least one protein disulfide isomerase or a biologically active fragment or variant thereof is recombinantly produced.

Illustrative embodiment 3. The pharmaceutical composition of Illustrative embodiment 1 or 2, further comprising at least one additional active agent selected from Heat Shock Protein 90 (HSP90), insulin, and combinations thereof.

Illustrative embodiment 4. A method comprising administering to a patient in need thereof at least one pharmaceutical composition of any of Illustrative embodiments 1-3.

Illustrative embodiment 5. The method of Illustrative embodiment 4, further defined as a method of reducing blood glucose in the patient.

Illustrative embodiment 6. The method of Illustrative embodiment 4 or 5, further defined as a method of treating hyperglycemia.

Illustrative embodiment 7. The method of any one of Illustrative embodiments 4-6, further defined as a method of treating or reducing the occurrence of diabetes in a patient.

Illustrative embodiment 8. The method of Illustrative embodiment 7, wherein the patient has or is predisposed to type 1 diabetes.

Illustrative embodiment 9. The method of Illustrative embodiment 7 or 8, wherein the patient has or is predisposed to type 2 diabetes.

Illustrative embodiment 10. The method of any one of Illustrative embodiments 7-9, wherein the patient has or is predisposed to type 3 diabetes (i.e., Alzheimer's disease).

Illustrative embodiment 11. The method of any of Illustrative embodiments 4-10, further defined as a method of treating or reducing the occurrence of Alzheimer's disease.

Illustrative embodiment 12. The method of any one of Illustrative embodiments 4-11, further defined as a method of improving glucose tolerance in the patient.

Illustrative embodiment 13. The method of any one of Illustrative embodiments 4-12, wherein the composition is administered intranasally.

Illustrative embodiment 14. A method of treating or reducing the occurrence of diabetes in a patient, the method comprising: administering to the patient at least one composition comprising a protein disulfide isomerase or a biologically active fragment or variant thereof.

Illustrative embodiment 15. The method of Illustrative embodiment 14, wherein the patient has or is predisposed to type 1 diabetes.

Illustrative embodiment 16. The method of Illustrative embodiment 14 or 15, wherein the patient has or is predisposed to type 2 diabetes.

Illustrative embodiment 17. The method of any one of Illustrative embodiments 14-16, wherein the patient has or is predisposed to type 3 diabetes (i.e., Alzheimer's disease).

Illustrative embodiment 18. The method of any of Illustrative embodiments 14-17, further defined as a method of treating or reducing the occurrence of Alzheimer's disease.

Illustrative embodiment 19. The method of any of Illustrative embodiments 14-18, further defined as a method of treating hyperglycemia in the patient.

Illustrative embodiment 20. The method of any of Illustrative embodiments 14-19, further defined as a method of reducing blood glucose in the patient.

Illustrative embodiment 21. The method of any of Illustrative embodiments 14-20, further defined as a method of improving glucose tolerance in the patient.

Illustrative embodiment 22. The method of any of Illustrative embodiments 14-21, wherein the at least one composition is administered intranasally.

Illustrative embodiment 23. The method of any of Illustrative embodiments 14-22, further comprising the step of administering at least one additional composition to the patient, wherein the two compositions are administered simultaneously or wholly or partially sequentially, and wherein the at least one additional composition comprises at least one active agent selected from the group consisting of HSP90, insulin, and combinations thereof.

Illustrative embodiment 24. A pharmaceutical composition for use in the method of any of Illustrative embodiments 14-23.

Illustrative embodiment 25. The pharmaceutical composition of Illustrative embodiment 24, wherein the pharmaceutical composition further comprises at least one pharmaceutically acceptable carrier.

Illustrative embodiment 26. The pharmaceutical composition of Illustrative embodiment 24 or 25, further comprising at least one additional active agent selected from Heat Shock Protein 90 (HSP90), insulin, and combinations thereof.

Illustrative embodiment 27. A kit, comprising at least one pharmaceutical composition of any of Illustrative embodiments 1-3 or 24-26.

Illustrative embodiment 28. The kit of Illustrative embodiment 27, further comprising at least one additional composition, and wherein the at least one additional composition comprises at at least one active agent selected from HSP90, insulin, and combinations thereof.

Thus, in accordance with the present disclosure, there have been provided devices, kits, and assemblies, as well as methods of producing and using same, which fully satisfy the objectives and advantages set forth herein. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

### REFERENCES

The following references, to the extent that they provide exemplary procedural or other details supplementary to those set forth herein, are specifically incorporated herein by reference. In addition, the following is not intended to be an Information Disclosure Statement; rather, an Information Disclosure Statement in accordance with the provisions of 37 CFR § 1.97 will be submitted separately.
(1) The Pathophysiology of Cardiovascular Disease and Diabetes: Beyond Blood Pressure and Lipids|Diabetes Spectrum|American Diabetes Association.
(2) Maria, Z., Campolo, A. R., and Lacombe, V. A. (2015) Diabetes Alters the Expression and Translocation of the Insulin-Sensitive Glucose Transporters 4 and 8 in the Atria. PLOS ONE 10, e0146033.
(3) Maria, Z., Campolo, A. R., Scherlag, B. J., Ritchey, J. W., and Lacombe, V. A. (2018) Dysregulation of insulin-sensitive glucose transporters during insulin resistance-induced atrial fibrillation. Biochim. Biophys. Acta Mol. Basis Dis. 1864, 987-996.
(4) Parriman, M., Campolo, A., Waller, A. P., and Lacombe, V. A. (2019) Adverse Metabolic Effects of Diltiazem Treatment During Diabetic Cardiomyopathy. J. Cardiovasc. Pharmacol. Ther. 24, 193-203.
(5) Waller, A. P., Kalyanasundaram, A., Hayes, S., Periasamy, M., and Lacombe, V. A. (2015) Sarcoplasmic reticulum Ca2+ ATPase pump is a major regulator of glucose transport in the healthy and diabetic heart. Biochim. Biophys. Acta 1852, 873-881.
(6) Periasamy, M., and Huke, S. (2001) SERCA pump level is a critical determinant of Ca(2+)homeostasis and cardiac contractility. J. Mol. Cell. Cardiol. 33, 1053-1063.
(7) Periasamy, M., and Kalyanasundaram, A. (2007) SERCA pump isoforms: their role in calcium transport and disease. Muscle Nerve 35, 430-442.
(8) Rochowski, M., Maria, Z., Campolo, A., Shoop, S., Hartson, S. D., Lopaschuk, G. D., and Lacombe, V. A. (2020) Abstract 15747: The Sarcoplasmic Reticulum Calcium ATPase Pump Modulates Whole-body Glucose Homeostasis Through a Protein Disulfide Isomerase-dependent Pathway. Circulation 142, A15747-A15747.
(9) Kranz, P., Neumann, F., Wolf, A., Classen, F., Pompsch, M., Ocklenburg, T., Baumann, J., Janke, K., Baumann, M., Goepelt, K., Riffkin, H., Metzen, E., and Brockmeier, U. (2017) PDI is an essential redox-sensitive activator of PERK during the unfolded protein response (UPR). Cell Death Dis. 8, e2986-e2986.
(10) Victor, P., Sarada, D., and Ramkumar, K. M. (2021) Crosstalk between endoplasmic reticulum stress and oxidative stress: Focus on protein disulfide isomerase and endoplasmic reticulum oxidase 1. Eur. J. Pharmacol. 892, 173749.
(11) Ma, J., and Hart, G. W. (2014) O-GlcNAc profiling: from proteins to proteomes. Clin. Proteomics 11, 8.
(12) Zeidan, Q., and Hart, G. W. (2010) The intersections between O-GlcNAcylation and phosphorylation: implications for multiple signaling pathways. J. Cell Sci. 123, 13-22.
(13) Wulff-Fuentes, E., Berendt, R. R., Massman, L., Danner, L., Malard, F., Vora, J., Kahsay, R., and Olivier-Van Stichelen, S. (2021) The human O-GlcNAcome database and meta-analysis. Sci. Data 8, 25.
(14) Vosseller, K., Wells, L., Lane, M. D., and Hart, G. W. (2002) Elevated nucleocytoplasmic glycosylation by O-GlcNAc results in insulin resistance associated with defects in Akt activation in 3T3-L1 adipocytes. Proc. Natl. Acad. Sci. U.S.A 99, 5313-5318.
(15) Macauley, M. S., Shan, X., Yuzwa, S. A., Gloster, T. M., and Vocadlo, D. J. (2010) Elevation of Global O-GlcNAc in Rodents Using a Selective O-GlcNAcase Inhibitor Does Not Cause Insulin Resistance or Perturb Glucohomeostasis. Chem. Biol. 17, 949-958.
(16) Bond, M. R., and Hanover, J. A. (2013)O-GlcNAc cycling: a link between metabolism and chronic disease. Annu. Rev. Nutr. 33, 205-229.
(17) Ong, Q., Han, W., and Yang, X. (2018)O-GlcNAc as an Integrator of Signaling Pathways. Front. Endocrinol. 9.
(18) Park, S. Y., Ryu, J., and Lee, W. (2005)O-GlcNAc modification on IRS-1 and Akt2 by PUGNAc inhibits their phosphorylation and induces insulin resistance in rat primary adipocytes. Exp. Mol. Med. 37, 220-229.
(19) Lu, S., Liao, Z., Lu, X., Katschinski, D. M., Mercola, M., Chen, J., Heller Brown, J., Molkentin, J. D., Bossuyt, J., and Bers, D. M. (2020) Hyperglycemia Acutely Increases Cytosolic Reactive Oxygen Species via O-linked GlcNAcylation and CaMKII Activation in Mouse Ventricular Myocytes. Circ. Res. 126, e80-e96.
(20) Nitric oxide synthases: regulation and function—PMC.
(21) Król, M., and Kepinska, M. (2020) Human Nitric Oxide Synthase—Its Functions, Polymorphisms, and Inhibitors in the Context of Inflammation, Diabetes and Cardiovascular Diseases. Int. J. Mol. Sci. 22, 56.
(22) Ragoobirsingh, D., McGrowder, D., Dasgupta, T., and Brown, P. (2004) The effect of nitric oxide on glucose metabolism. Mol. Cell. Biochem. 263, 29-34.
(23) Bahadoran, Z., Mirmiran, P., and Ghasemi, A. (2020) Role of Nitric Oxide in Insulin Secretion and Glucose Metabolism. Trends Endocrinol. Metab. 31, 118-130.
(24) Aasum, E., Hafstad, A. D., Severson, D. L., and Larsen, T. S. (2003) Age-dependent changes in metabolism, contractile function, and ischemic sensitivity in hearts from db/db mice. Diabetes 52, 434-441.
(25) Lund, J., Hafstad, A. D., Boardman, N. T., Rossvoll, L., Rolim, N. P., Ahmed, M. S., Florholmen, G., Attramadal, H., Wisløff, U., Larsen, T. S., and Aasum, E. (2015) Exercise training promotes cardioprotection through oxygen-sparing action in high fat-fed mice. Am. J. Physiol. Heart Circ. Physiol. 308, H823-829.
(26) Barr, R. L., and Lopaschuk, G. D. (1997) Direct measurement of energy metabolism in the isolated working rat heart. J. Pharmacol. Toxicol. Methods 38, 11-17.
(27) Schmittgen, T. D., and Livak, K. J. (2008) Analyzing real-time PCR data by the comparative C (T) method. Nat. Protoc. 3, 1101-1108.
(28) Maria, Z., and Lacombe, V. A. (2018) Quantification of Cell-Surface Glucose Transporters in the Heart Using a Biotinylated Photolabeling Assay. Methods Mol. Biol. Clifton NJ 1713, 229-240.
(29) Merlet, N., Piriou, N., Rozec, B., Grabherr, A., Lauzier, B., Trochu, J.-N., and Gauthier, C. (2013) Increased Beta2-Adrenoceptors in Doxorubicin-Induced Cardiomyopathy in Rat. PLOS ONE 8, e64711.
(30) Birenbaum, A., Tesse, A., Loyer, X., Michelet, P., Andriantsitohaina, R., Heymes, C., Riou, B., and Amour, J. (2008) Involvement of β3-Adrenoceptor in Altered β-Adrenergic Response in Senescent Heart: Role of Nitric Oxide Synthase 1-derived Nitric Oxide. Anesthesiology 109, 1045-1053.
(31) Rajpal, G., Schuiki, I., Liu, M., Volchuk, A., and Arvan, P. (2012) Action of Protein Disulfide Isomerase on Proinsulin Exit from Endoplasmic Reticulum of Pancreatic β-Cells. J. Biol. Chem. 287, 43-47.
(32) Ferron, M., Cadiet, J., Persello, A., Prat, V., Denis, M., Erraud, A., Aillerie, V., Mevel, M., Bigot, E., Chatham, J. C., Gauthier, C., Rozec, B., and Lauzier, B. (2019)O-GlcNAc stimulation: A new metabolic approach to treat septic shock. Sci. Rep. 9, 18751.
(33) Wright, J. N., Collins, H. E., Wende, A. R., and Chatham, J. C. (2017)O-GlcNAcylation and cardiovascular disease. Biochem. Soc. Trans. 45, 545-553.
(34) Volpe, C. M. O., Villar-Delfino, P. H., dos Anjos, P. M. F., and Nogueira-Machado, J. A. (2018) Cellular death, reactive oxygen species (ROS) and diabetic complications. Cell Death Dis. 9, 119.
(35) Gheibi, S., Samsonov, A. P., Gheibi, S., Vazquez, A. B., and Kashfi, K. (2020) Regulation of carbohydrate metabolism by nitric oxide and hydrogen sulfide: Implications in diabetes. Biochem. Pharmacol. 176, 113819.
(36) Ali Khan, H., and Mutus, B. (2014) Protein disulfide isomerase a multifunctional protein with multiple physiological roles. Front. Chem. 2.
(37) Fliegel, L., Newton, E., Burns, K., and Michalak, M. (1990) Molecular cloning of cDNA encoding a 55-kDa multifunctional thyroid hormone binding protein of skeletal muscle sarcoplasmic reticulum. J. Biol. Chem. 265, 15496-15502.
(38) Hashimoto, S., and Imaoka, S. (2013) Protein-disulfide isomerase regulates the thyroid hormone receptor-mediated gene expression via redox factor-1 through thiol reduction-oxidation. J. Biol. Chem. 288, 1706-1716.
(39) Maeda, R., Ado, K., Takeda, N., and Taniguchi, Y. (2007) Promotion of insulin aggregation by protein disulfide isomerase. Biochim. Biophys. Acta 1774, 1619-1627.
(40) Wang, Q., Fang, P., He, R., Li, M., Yu, H., Zhou, L., Yi, Y., Wang, F., Rong, Y., Zhang, Y., Chen, A., Peng, N., Lin, Y., Lu, M., Zhu, Y., Peng, G., Rao, L., and Liu, S. (2020)O-GlcNAc transferase promotes influenza A virus-induced cytokine storm by targeting interferon regulatory factor-5. Sci. Adv. 6, eaaz7086.
(41) Gonzalez-Rellan, M. J., Fondevila, M. F., Dieguez, C., and Nogueiras, R. (2022)O-GlcNAcylation: A Sweet Hub in the Regulation of Glucose Metabolism in Health and Disease. Front. Endocrinol. 13.
(42) Harris, M. B., Mitchell, B. M., Sood, S. G., Webb, R. C., and Venema, R. C. (2008) Increased nitric oxide synthase activity and Hsp90 association in skeletal muscle following chronic exercise. Eur. J. Appl. Physiol. 104, 795-802.
(43) Bell, S. E., Shah, C. M., and Gordge, M. P. (2007) Protein disulfide-isomerase mediates delivery of nitric oxide redox derivatives into platelets. Biochem. J. 403, 283-288.
(44) Hofmann, H., and Schmidt, H. H. H. W. (1995) Thiol Dependence of Nitric Oxide Synthase. Biochemistry 34, 13443-13452.
(45) High Glucose Increases Nitric Oxide Synthase Expression and Superoxide Anion Generation in Human Aortic Endothelial Cells|Circulation.
(46) Hulme, K. D., Gallo, L. A., and Short, K. R. (2017) Influenza Virus and Glycemic Variability in Diabetes: A Killer Combination? Front. Microbiol. 8, 861.

1. A pharmaceutical composition, comprising:
at least one protein disulfide isomerase or a biologically active fragment or variant thereof; and
at least one pharmaceutically acceptable carrier.

2. The pharmaceutical composition of claim 1, wherein the at least one protein disulfide isomerase or a biologically active fragment or variant thereof is recombinantly produced.

3. The pharmaceutical composition of claim 1, further comprising at least one additional active agent selected from Heat Shock Protein 90 (HSP90), insulin, and combinations thereof.

4. A method comprising:
administering at least one pharmaceutical composition to a patient in need thereof, wherein the at least one pharmaceutical composition comprises:
at least one protein disulfide isomerase or a biologically active fragment or variant thereof; and
at least one pharmaceutically acceptable carrier.

5. The method of claim 4, further defined as a method of reducing blood glucose in the patient.

6. The method of claim 4, further defined as a method of treating hyperglycemia.

7. The method of claim 4, further defined as a method of treating or reducing the occurrence of diabetes in a patient.

8. The method of claim 7, wherein the patient has or is predisposed to at least one of type 1 diabetes, type 2 diabetes, and/or type 3 diabetes.

9. The method of claim 4, further defined as a method of improving glucose tolerance in the patient.

10. The method of claim 4, further defined as a method of treating or reducing the occurrence of Alzheimer's disease.

11. The method of claim 4, wherein the composition is administered intranasally.

12. The method of claim 4, further comprising the step of administering at least one additional composition to the patient, wherein the two compositions are administered simultaneously or wholly or partially sequentially, and wherein the at least one additional composition comprises at least one active agent selected from the group consisting of HSP90, insulin, and combinations thereof.

13. A method of treating or reducing the occurrence of diabetes in a patient, the method comprising:
administering to the patient at least one composition comprising a protein disulfide isomerase or a biologically active fragment or variant thereof.

14. The method of claim 13, wherein the patient has or is predisposed to type 1 diabetes.

15. The method of claim 13, wherein the patient has or is predisposed to type 2 diabetes.

16. The method of claim 13, wherein the patient has or is predisposed to type 3 diabetes.

17. The method of claim 13, wherein the composition is administered intranasally.

18. The method of claim 13, further comprising the step of administering at least one additional composition to the patient, wherein the two compositions are administered simultaneously or wholly or partially sequentially, and wherein the at least one additional composition comprises at least one active agent selected from the group consisting of HSP90, insulin, and combinations thereof.

19. A kit, comprising:
at least one pharmaceutical composition of claim 1; and
at least one additional composition, wherein the at least one additional composition comprises at least one active agent selected from HSP90, insulin, and combinations thereof.
